(19) United States
(12) Patent Application Publication
(10) Pub. No.: US 2014/0172006 A1
(43) Pub. Date: Jun. 19, 2014

(54) SYSTEM FOR FACILITATING TRANSCATHETER AORTIC VALVE PROCEDURES USING FEMORAL ACCESS

(71) Applicant: Synecor LLC, Chapel Hill, NC (US)

(72) Inventors: Richard S. Stack, Chapel Hill, NC (US); Richard A. Glenn, Chapel Hill, NC (US); Michael S. Williams, Santa Rosa, CA (US); William L. Athas, Chapel Hill, NC (US); Colleen Stack, Chapel Hill, NC (US)

(21) Appl. No.: 13/975,331

(22) Filed: Aug. 24, 2013

Related U.S. Application Data
(60) Provisional application No. 61/692,704, filed on Aug. 24, 2012.
(60) Provisional application No. 61/703,185, filed on Sep. 19, 2012.
(60) Provisional application No. 61/728,679, filed on Nov. 20, 2012.

(51) Int. Cl. A61F 2/01

(52) U.S. Cl. CPC A61F 2/013
USPC 606/200

(57) ABSTRACT

A device for facilitating use of instruments disposed through an aortic arch includes an embolic deflector having a first surface positionable in contact with a wall of an aortic arch such that a porous barrier portion of the embolic deflector covers ostia of at least the brachiocephalic and left common carotid arteries. A second surface is disposed on an opposite face from the first surface. A lubricious guide track is disposed on the second surface and extends longitudinally on the embolic deflector. The deflector and/or guide track is supported by a shaft that is extendable through a femoral artery and descending aorta to position the guide within the aortic arch. During use, the device is percutaneously introduced via the femoral artery and advanced into the aorta. The porous barrier portion of the deflector is positioned over the target ostia, and the guide track thus faces into the aortic arch. An instrument passed through the aortic arch, such as an aortic valve delivery system introduced via a femoral artery, is advanced along the lubricious guide track towards a target site (e.g. the aortic valve), minimizing contact between the instrument and the wall of the aorta.

This application claims the benefit of U.S. Provisional Application No. 61/692,704, filed 24 Aug. 2012, U.S. Provisional Application No. 61/703,185, filed 19 Sep. 2012, and U.S. Provisional No. 61/728,679, filed 20 Nov. 2012, each of which is incorporated herein by reference.

### FIELD OF THE INVENTION

The field of the invention relates generally to the field of devices used to facilitate catheter-based procedures in which instruments are positioned through or within the aorta, such as for treatment of the aortic valve or replacement of the valve.

### BACKGROUND

Transcatheter aortic-valve implantation (TAVI) has emerged as a therapeutic option to improve symptoms and extend life in high-risk patients with severe symptomatic Aortic Stenosis.

One TAVI approach is a transfemoral (TF) route in which catheters are introduced into the femoral artery and passed into the aorta via the descending aorta. The catheters are guided through the aorta and retrograde across the diseased valve.

When instruments are advanced through the aorta, care must be taken to avoid embolization that might occur as instruments are passed along the curvature of the aortic arch. In particular, embolic material can be dislodged from the wall of the aortic arch as catheters or other instruments are passed along the arch. The disclosed system provides an access track allowing catheters and other instruments to move through the arch with minimal wall contact, so as to minimize the likelihood that embolic material will be released from the wall of the arch. In the illustrated embodiments, the access track is positioned on an embolic deflector device, such that any embolic material released during performance of a procedure using the system may be diverted away from the arterial vessels leading into the head.

### BRIEF DESCRIPTION OF THE DRAWINGS

FIGS. 1 and 2 are perspective views of a first embodiment of an embolic deflector and guide device.

FIG. 3 schematically illustrates the embolic deflector and guide device of FIGS. 1 and 2 within an aorta.

FIG. 4 is similar to FIG. 3, and further shows a procedure device and pigtail catheter in use with the embolic deflector and guide device.

FIGS. 5 and 6 are similar to FIGS. 3 and 4 and show an alternative embodiment of the embolic deflector and guide device.

FIGS. 7 and 8 are similar to FIGS. 3 and 4 and show another alternative embodiment of the embolic deflector and guide device.

### DETAILED DESCRIPTION

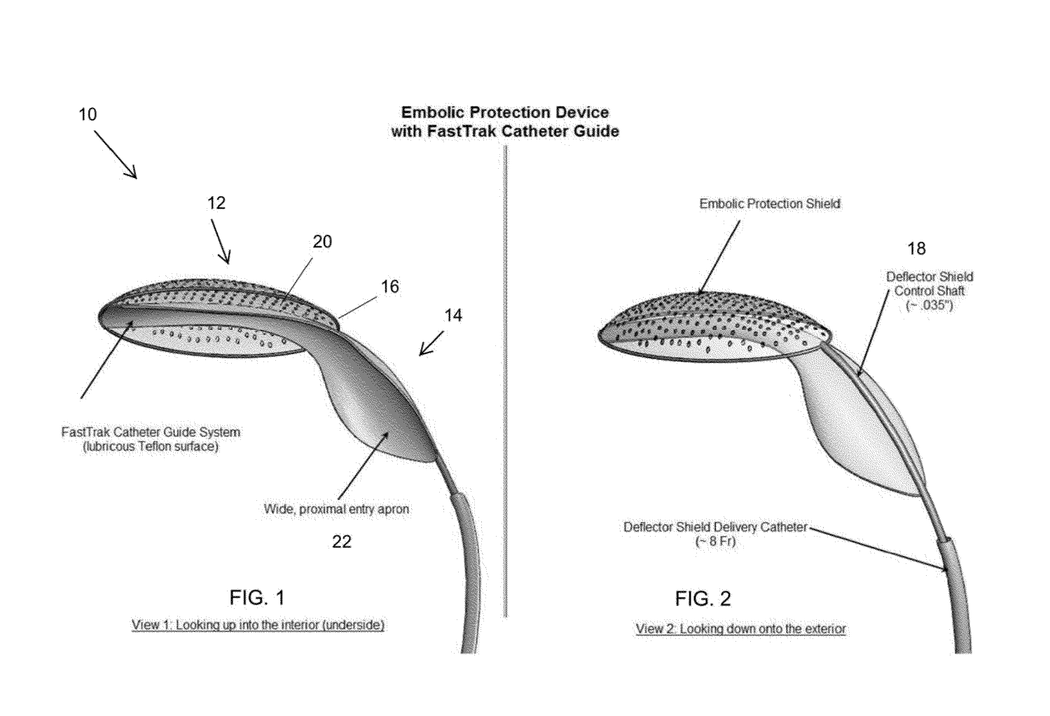

FIGS. 1 and 2 show a first embodiment of an embolic deflector and guide device 10. Device includes a deflector portion 12 and a guide 14.

The deflector portion 12 is formed of a flexible frame 16 defining an open area. The frame is preferably made of nitinol or similar material, and is shape set to the desired shape.

An elongate control/support shaft 18 or wire extends from the proximal portion of the frame. The support shaft can be a separate element that is attached to the frame or the frame and support may be formed of one continuous wire during heat setting, thus removing the need to connect or couple the frame to the shaft.

A barrier 20 is supported by the frame 16, along its perimeter. The barrier is one that will prevent passage of emboli through it, but at least certain regions of the barrier are porous so as to allow blood to flow through it. In one embodiment, the porous barrier may be formed of porous silicone or polyurethane, or other materials such as woven materials. In one embodiment, the covering may be applied using dip, molding and/or spray techniques. The barrier preferably contacts the full inner perimeter of the frame, but in some embodiments the outer perimeter of the frame may be formed to be free of the barrier material to facilitate sliding of the deflector within the delivery and removal catheter(s).

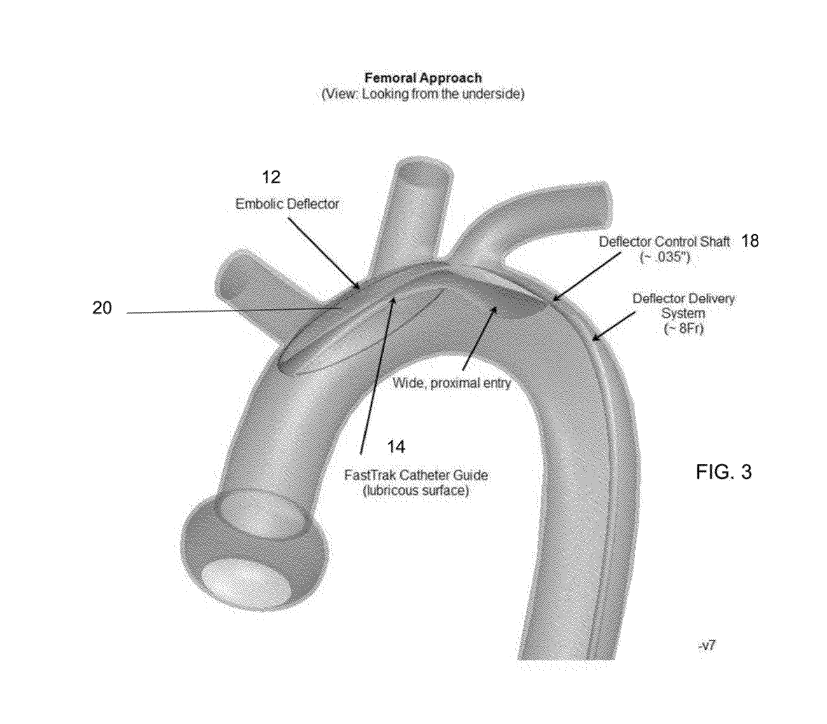

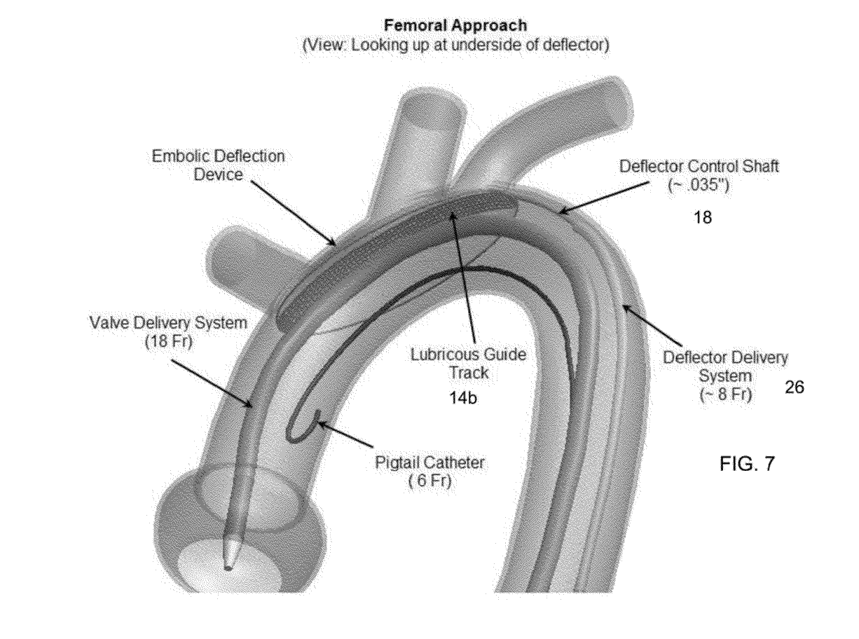

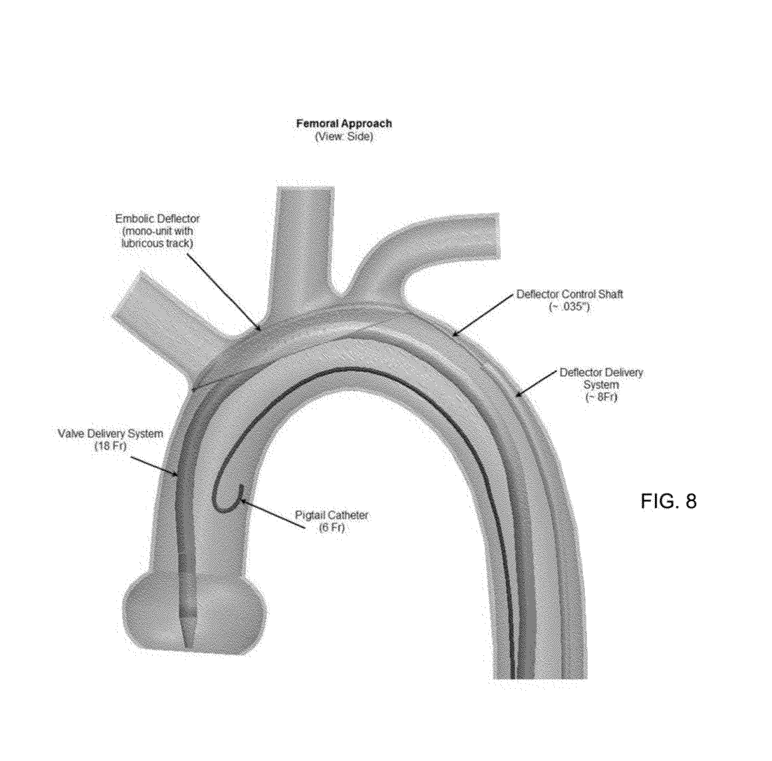

As shown in FIG. 3, the barrier 20 has sufficient distal-to-proximal length to cover the ostia of the brachiocephalic artery (through which blood flows into the right subclavian and right common carotid arteries) and the left common carotid artery. In other embodiments (such as the FIGS. 7 and 8 embodiment), the length may be sufficient to also cover the ostium of the left subclavian artery.

The embolic diverter may be formed to have a variety of shapes. In the illustrated embodiment, the frame and barrier define a generally oval shape. The curvature of the diverter is selected to approximately track the curvature of the portion of the aortic wall along which the target ostia are position, e.g. the surface of the barrier that faces into the aortic arch is concave, and the surface contacting the wall of the aorta and covering the ostia is convex. This positions the barrier away from the lumen of the aortic arch so it will be less likely to obstruct blood flow within the arch or the passage of instruments through the arch.

Additional details of embolic deflector devices that can be adapted for use with the disclosed system are shown and described in U.S. application Ser. No. 13/773,625, filed Feb. 21, 2013, entitled Embolic Protection System and Method for Use in an Aortic Arch, which is incorporated herein by reference.

The guide 14 of the device 10 is positioned on the surface of the barrier that faces into the aorta. Guide 14 functions as a track along which instruments 100 passing into the aortic arch from the descending aorta can slide. In the FIGS. 1 and 2 embodiment, guide 14 includes a broad entry apron 22 carried by the shaft 18 and disposed proximal to the frame 16, providing a wide landing area for a catheter moving into the aortic arch from the descending aorta. The portion of the guide 14 located on the concave surface of the barrier 20 may be more narrow—thus minimizing obstruction of the blood flow pores/openings in the barrier 20. In other words, the lateral dimension of the guide 14 (extending perpendicular to the longitudinal axis defined by the shaft 18) is greater at the entry apron than along the barrier. As shown, the guide 14 has a concave shape, forming a channel having wall portions to urge an instrument 100 passing along the track towards the longitudinal center of the track—thus minimizing the chance that the instrument will slip laterally over the banks of the track. The contact surface of the track (the surface along which the instrument slides) includes a lubricious surface formed of Teflon or other lubricious material.

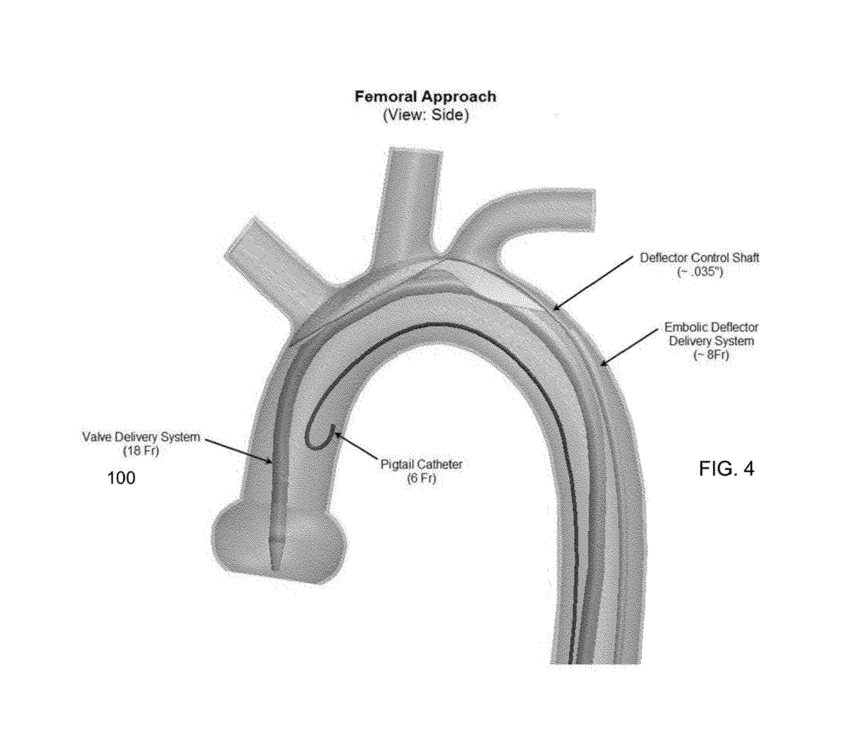

In the first embodiment, the length of the guide 14 in the proximal direction extends past the left subclavian artery as shown in FIG. 4, preferably to a point where the proximal end of the guide 14 curves downwardly towards or into the descending aorta, facilitating the process of landing the instrument 100 onto the track as the instrument is guided from the descending aorta towards the track. With this positioning, the guide may also helps divert any embolic material away from the left subclavian artery.

The guide may be formed of a material or combination of materials that allow the guide to be collapsible into a catheter for deployment, but that will give sufficient strength to the guide to maintain its shape during use. Exemplary materials include PTFE, ePTFE, lubricated silicone or urethane. These materials might be provided as sheets or membranes mounted to or formed on nitinol or stainless steel frame having the desired shape (possibly similar in construction to the frame that supports the barrier). In another embodiment, the track might be a thin film-like sheet of nitinol that has been shape-set into the desired shape. In yet another embodiment, the track may be formed using a thin-walled balloon inflating using saline once it has been positioned within the aorta. The balloon is deflated by withdrawing the saline or perforating the balloon prior to withdrawal.

In use, the embolic deflector and guide device 10 is disposed within a catheter 26 and introduced into the vasculature through an access port in the femoral artery, with the proximal end of the shaft 18 extending out of the body. The distal end of the catheter 26 is advanced through the descending aorta and positioned (using the control shaft 18 and/or catheter 26) with its distal opening upstream of the brachocephalic artery. The embolic deflector and guide device is deployed from the catheter 26, causing the frame to expand. The expanded frame preferably contacts the surrounding walls of the aortic arch.

In the FIG. 1-6 embodiments, upon deployment of the device 10, the distal end of the barrier 20 is positioned upstream of the ostium of the brachiocephalic artery, and the proximal end the deflector is positioned downstream of the ostium of the left common carotid artery. In other embodiments (including the FIG. 7-8 embodiment), the proximal end of the deflector is deployed to a position downstream of the left subclavian artery.

Next, an instrument 100 used to perform a procedure is introduced through the femoral artery and advanced into the descending aorta. In the drawings, instrument 100 is shown as a delivery system for a transcatheter aortic valve replacement procedure, although the system will accommodate other types of instruments. Instrument 100 is guided into contact with the entry apron 22. Depending on the orientation of the instrument 100, its tip may be the first part of the instrument to contact the entry apron 22.

As the instrument 100 is further advanced along the guide 14 towards the aortic root, the guide's banked walls contain the instrument against slipping laterally off the guide. The instrument 100 may remain in contact with the guide 14 throughout the valve replacement or other procedure; minimizing the likelihood that contact between the instrument 100 and the wall of the aortic arch will release embolic material. Emboli may nevertheless be released into the aorta during the procedure, particularly as the stenotic valve is treated. Any such emboli will be unable to pass into the brachocephalic and left common carotid arteries due to the presence of the barrier 20 of the deflector 12 covering the entrances to those arteries. Such emboli will thus bypass the ostia of the covered vessels and exit the aortic arch through the descending aorta.

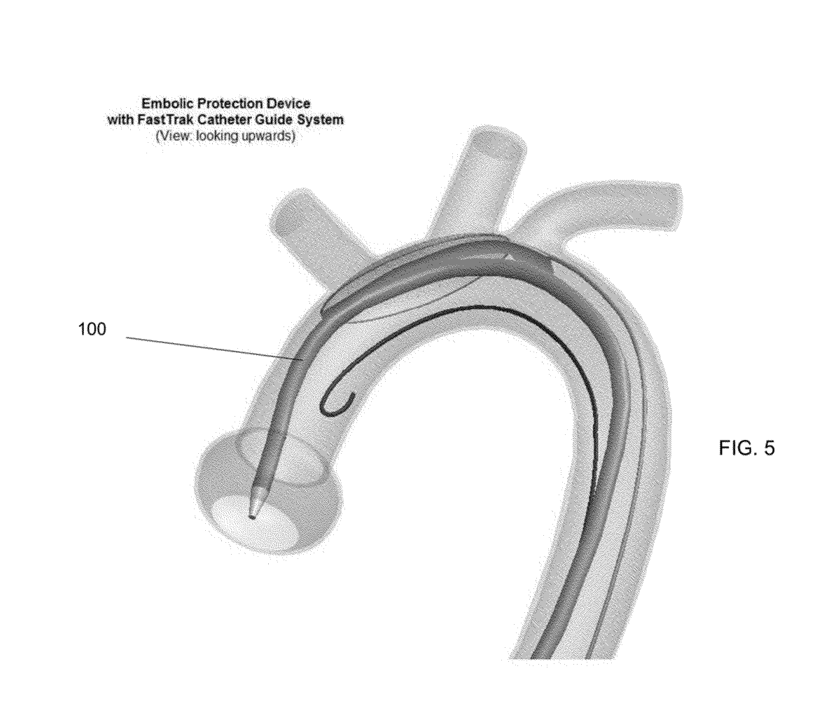

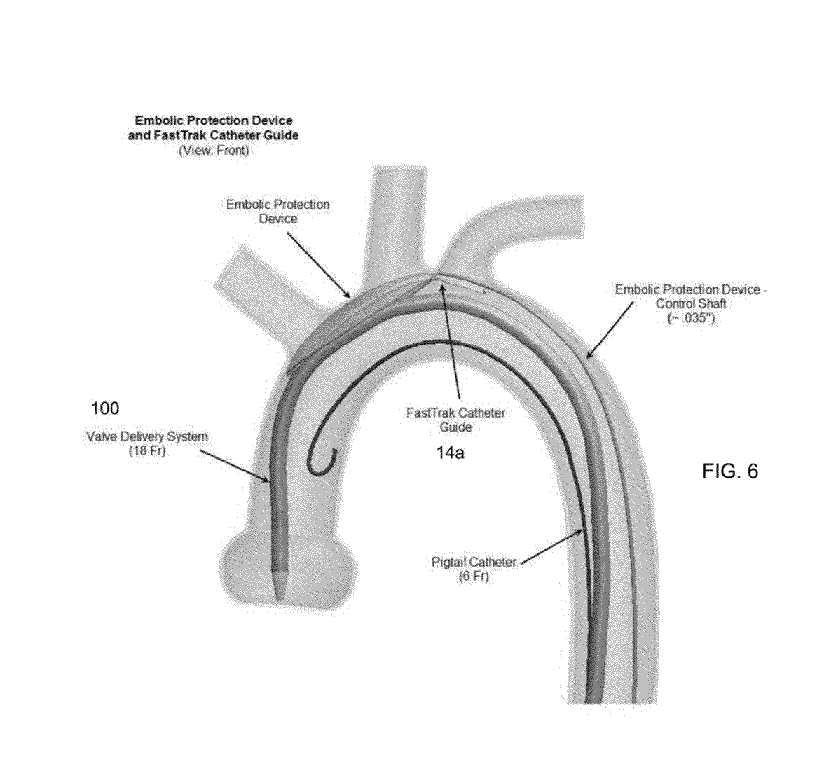

In a first alternate embodiment shown in FIGS. 5 and 6, the entry apron 22a of the guide 14a has a smaller width and shorter length than the guide of the first embodiment.

In a second alternate embodiment shown in FIGS. 7 and 9, the guide 14b is provided without an entry apron. In this and other embodiments, the guide 14b may be provided without walls on either side of the longitudinal axis, but might be instead be formed as a lubricious strip along the surface of the barrier of the deflector.

Although the deflector and guide have been described of elements of a unitary device, in alternate embodiments the deflector and guide may be separate components of a system. In such embodiments, the deflector and guide might be separately deployable, separately deployable but engageable with one another within the aorta, or provided separately and engageable with one another prior to deployment.

All prior patents and patent applications referred to herein, including for purposes of priority, are incorporated herein by reference.

We claim:

1. A method for performing a procedure using instruments disposed through an aortic arch, comprising the steps of:
positioning a lubricious track within an aortic arch;
percutaneously introducing an instrument into a femoral artery and advancing the instrument through a descending aorta into the aortic arch and into contact with the track;
with the instrument in contact with the track, advancing a distal portion of the instrument to the target operative site; and
performing a procedure at the operative site using the instrument.

2. The method of claim 1, wherein track includes a longitudinal axis and wall portions on opposite sides of the longitudinal axis defining a channel, wherein advancing the instrument into contact with the track includes advancing the instrument along the channel.

3. The method of claim 1, further including introducing an embolic deflector into the aorta and positioning a barrier of the embolic deflector covering ostia of at least a brachiocephalic artery and a left common carotid artery, wherein the deflector includes a convex surface contacting the aortic arch, and a concave surface facing into the lumen, and wherein at least a portion of the track contacts the concave surface of the deflector.

4. The method of claim 3, wherein the track is carried by the deflector, and wherein the step of positioning the barrier includes the step of positioning the track within the aortic arch.

5. A device for facilitating use of instruments disposed through an aortic arch, the device comprising:
a guide having a longitudinal axis and wall portions extending along opposite sides of the longitudinal axis to define a track;
a lubricious surface on the track;
a shaft supporting the guide, the shaft extendable through a femoral artery and descending aorta to position the guide within an aortic arch.

6. The device of claim 5, further including a deflector having a barrier positionable covering ostia of at least a brachiocephalic artery and a left common carotid artery, wherein the guide is positionable in contact with the deflector.

7. The device of claim 6, wherein the deflector includes a convex surface contacting the aortic arch, and a concave surface facing into the lumen, and wherein at least a portion of the track contacts the concave surface of the deflector.

8. The device of claim 7, wherein the track is coupled to the concave surface of the deflector.

9. The device of claim 8, wherein the track includes a proximal portion extending proximally of the barrier and a distal portion contacting the deflector.

10. The device of claim 9, where the track is wider, relative to the longitudinal axis, in the proximal portion than in the distal portion.

11. A device for facilitating use of instruments disposed through an aortic arch, the device comprising:
an embolic deflector having
a first surface positionable in contact with a wall of an aortic arch such that at least a portion of the embolic deflector covers ostia of at least the brachiocephalic and left common carotid arteries, and
a second surface on an opposite face from the first surface;
a lubricious guide track disposed on the second surface and extending longitudinally from a proximal portion to a distal portion of the embolic deflector; and
a shaft supporting at least one of the deflector and guide track, the shaft extendable through a femoral artery and descending aorta to position the guide within an aortic arch.

12. The device of claim 11, wherein the guide track includes a longitudinal axis and wall portions on opposite sides of the longitudinal axis, defining a channel.
